(19) United States
(12) Patent Application Publication
(10) Pub. No.: US 2003/0171260 A1
(43) Pub. Date: Sep. 11, 2003

(54) Compositions and methods utilizing hydroxamates to scavenge oxidant toxins

(76) Inventor: Deanna Jean Nelson, Cary, NC (US)

Correspondence Address: Deanna J. Nelson, Ph.D., 104 Tasman Court, Cary, NC, 27513, US

(21) Appl. No.: 10/095,740

(22) Filed: Mar. 11, 2002

(51) Int. Cl. A61K 38/16; A01N 1/00

(52) U.S. Cl. 514/6; 435/1.1

(57) ABSTRACT

Macromolecule compositions using hydroxamate or oxime groups covalently coupled with biological cells and macromolecules, and in particular, with albumin and acellular hemoglobin, are provided. In particular embodiments, hydroxamate or oxime groups are covalently coupled with acellular hemoglobin and compounded to provide several formulations for a red cell substitute that will possess oxidant toxin-scavenging properties. The invention also provides a method of preventing and treating pathologies related to the actions of oxidant toxins or oxidative stress in a mammal. The method comprises administering to a mammal a therapeutically effective amount of a macromolecule composition comprising a macromolecule coupled with at least one hydroxamate or oxime group.

### CROSS-REFERENCE TO RELATED APPLICATIONS

None.

### STATEMENT REGARDING FEDERALLY SPONSORED RESEARCH & DEVELOPMENT

No Federally sponsored research and development were used in making this invention.

### FIELD OF THE INVENTION

This invention relates to the use of hydroxamate and oxime groups that are covalently coupled with cells or macromolecules, including hemoglobin, albumin, polysaccharides, lipopolysaccharides, polynucleotides, liposomes, and natural and synthetic polymers, and have the ability to alleviate the toxic effects of oxidant toxins presumed to cause harmful oxidant stress in a living organism. In particular, this invention discloses compounds and methods featuring hydroxamate and oxime groups covalently coupled with physiologically compatible acellular and encapsulated hemoglobin solutions for use as a red cell substitute; and hydroxamate and oxime groups covalently coupled with other physiologically compatible macromolecules for alleviation and prevention of biological damage and oxidative stress caused by free radicals.

### BACKGROUND OF THE INVENTION

Although free radicals are produced continuously in cells, either as by-products of metabolism or by leakage from mitochondrial respiration, cells have developed a comprehensive set of antioxidant defense mechanisms to limit their damaging effects. However, when these defense mechanisms are compromised, perhaps by inflammation or disease states, elevated levels of reactive oxygen or nitrogen species such as the superoxide radical anion, the hydroxyl radical, and peroxynitrite may result. Chronic exposure to these oxidant toxins is known to damage biological systems. The oxidative stress that results can lead to oxidation of all cellular macromolecules, most notably lipids, proteins, and DNA; depletion of intracellular antioxidants and energy stores that would otherwise protect the cell; the release of intracellular stores of calcium and the inhibition of important cellular processes and signal transduction pathways that are essential to cell survival.

Establishing the involvement of these oxidant toxins in the pathogenesis of a disease, however, is extremely difficult, due to the short lifetimes of these species, and to the lack of sensitive and reliable markers of damaging exposure. Nonetheless, recent research has implicated both superoxide and peroxynitrite as damaging agents in a number of pathological inflammatory conditions, including ischemic injury, septic shock, chronic tissue rejection, multiple sclerosis, amyotrophic lateral sclerosis, Alzheimer's disease, cardiomyopathy, and atherosclerosis. Further, a number of diseases and disorders are now associated with chronic exposure to oxidative stress, including emphysema, arthritis, cancer, cataracts, and aging. Similarly, extreme inflammatory conditions that result from inappropriately prolonged systemic responses to acute invasion of the bloodstream by microorganisms or their toxic products, such as Acute Respiratory Distress Syndrome (ARDS), bacteremia, or septic shock, have been associated with oxidative stress related to these oxidant toxins.

Current therapeutic approaches to treating oxidative stress-related pathologies mitigate the biological effects of the oxidant toxins but do not prevent biological damage by inactivating these agents as they are formed in the body. Therefore, there is a clear and present need for the addition of persistent therapeutic agents that would inactivate the oxidant toxins themselves as they are formed, thereby preventing the subsequent biological effects of their actions. The present invention addresses this need.

A second and parallel need for therapeutic agents useful for preventing or treating oxidative stress-related pathologies is related to the development of red cell substitutes based on acellular hemoglobin. (See U.S. Pat. Nos. 3,925,344; 4,001,200; 4,001,401; 4,053,590; 4,061,736; 4,136,093; 4,301,144; 4,336,248; 4,376,095; 4,377,512; 4,401,652; 4,473,494; 4,473,496; 4,600,531; 4,584,130; 4,857,636; 4,826,811; 4,911,929; 5,061,688; and 5,843,888; 5,849,688; 5,895,810; 5,917,020; 5,952,482; 5,955,581; 5,962,651; 5,981,710; 6,022,544; 6,114,505; 6,124,114; 6,133,425; 6,150,507; 6,172,039; 6,184,356; and 6,235,500.) These Hemoglobin-Based Oxygen Carriers (HBOCs) are particularly attractive as red cell replacements because they avoid some of the shortcomings of red cells. However, to date, concerns about the toxicity of the modified hemoglobins that currently are being evaluated as blood substitutes have resulted in at least three clinical trials being halted and have significantly delayed regulatory approval of these products. A body of evidence suggests the observed toxicities are related, at least in part, to the actions of four oxidant toxins (superoxide, ferryl hemoglobin, the hydroxyl radical, and peroxynitrite) which are generated as hemoglobin circulates in the body outside the red cell. The origin of each of these species can be traced, at least in part, to the auto-oxidation of hemoglobin, discussed in greater detail below.

Auto-oxidation of Acellular Hemoglobin to Methemoglobin. The auto-oxidation of hemoglobin (Hb) to methemoglobin (MetHb) and superoxide is an important reaction that compromises the usefulness of acellular hemoglobin as a red cell substitute. The protein is administered and circulates for varying lengths of time as oxyhemoglobin. However, with time, the ferrous iron contained in each heme ligand undergoes either auto- or chemically induced oxidation, yielding methemoglobin. The hemoglobin auto-oxidation process, which applies to all hemoglobins, is summarized in Eq 1, and is accompanied by production of superoxide, hydroxyl radical and hydrogen peroxide.

4Hb(Fe⁺²—O₂)+2H⁺→4Hb(Fe⁺³)+2OH⁻+3O₂ (Eq. 1)

Hemoglobin oxidation has a number of potentially serious consequences for the patient. For example, in human erythrocytes, the methemoglobin reductase and other enzyme systems reduce MetHb to the functional, reduced form. However, both enzymatic and non-enzymatic methemoglobin reduction systems are virtually non-existent in the plasma. Thus, acellular methemoglobins are not reduced as they circulate throughout the body following infusion. In addition, methemoglobin does not carry oxygen. Thus, the efficacy of oxygen delivery continuously decreases as hemoglobin is oxidized to methemoglobin, perhaps before the red cell mass has been repleted sufficiently or the microcirculation has been restored. More seriously, oxidation to methemoglobin may be followed by hemichrome formation; loss of the heme moieties and their deposition on the cells lining the vascular system; protein denaturation; and other debilitating changes. Finally, other oxidation processes mediated by acellular hemoglobin can generate three other strong oxidants, ferryl hemoglobin, the hydroxyl radical, and peroxynitrite.

Superoxide Radical Anion Co-Generation. Auto-oxidation of the ferrous iron in each heme ligand on hemoglobin co-generates another potent toxin, the superoxide radical anion.

Hb(Fe⁺²—O₂)→Hb(Fe⁺³)+O₂⁻ (Eq. 2)

Superoxide is known to initiate acute tissue injury, the results of which are observed as oxidized lipids, hydroxylated aromatic residues in proteins or polynucleotides, and the formation of isoprostanes and other biomarkers. Moreover, superoxide is converted to hydrogen peroxide by the action of superoxide dismutase (SOD), a reaction that converts a relatively transient species to a more persistent one that is itself a potent oxidant toxin. Likewise, superoxide reacts at a nearly diffusion-controlled rate with nitric oxide to form the peroxynitrite anion. The toxic actions of peroxynitrite are summarized below.

Ferryl Hemoglobin Formation. The reaction between oxy or ferric forms of hemoglobin with hydrogen peroxide is known to proceed via the formation of a ferryl intermediate. Hydrogen peroxide is produced both by enzymatic dismutation of superoxide and from other biological sources (e.g., platelets, neutrophils, granulocytes, and macrophages activated by inflammatory mediators). Ferryl hemoglobin can peroxidize lipids, degrade carbohydrates, and modify proteins. Although ferryl hemoglobin is generally viewed as a transient species, investigators have shown that the ferryl form of some modified, acellular hemoglobins is more persistent.

Hydroxyl Radical Formation. Hemoglobin can catalyze a “Fenton” reaction in which the hydroxyl radical is formed from hydrogen peroxide. The hydroxyl radical is known to react at nearly diffusion-limited rates with any component in the cell, thereby initiating reactions that result in lipid peroxidation, carbohydrate degradation, DNA damage, and protein modification. The net result of this non-specific attack is a loss of cell integrity, enzyme function, and genomic stability.

Peroxynitrite-Forming Reactions. Peroxynitrite is a strong oxidant that can be formed by the nearly diffusion-controlled reaction of nitric oxide (NO) and superoxide (O₂⁻). The chemistry of peroxynitrite is currently being actively investigated. Already it has been shown that one- or two-electron oxidations by peroxynitrite (or its protonated counterpart, peroxynitrous acid) can damage DNA, initiate lipid peroxidation, or modify aromatic- or sulfur-containing amino acid residues. Peroxynitrite nitrates aromatic compounds in a reaction that can be catalyzed by metal complexes or metal-containing proteins. Moreover, peroxynitrite reacts rapidly with carbon dioxide to yield 1-carboylato-2-nitrosodioxidane, a stronger nitrating agent than peroxynitrite. Within seconds after it is formed, peroxynitrous acid spontaneously decomposes to .NO₂ and an oxidant with a reactivity similar to that of the hydroxyl radical.

Little progress to date has been made in mitigating or eliminating these oxidative stress-related toxicities of acellular hemoglobin. Thus, there is a clear and present need for persistent scavengers of these oxidant toxins that are generated when an acellular hemoglobin is present in the systemic circulation. The present invention also addresses this need.

Before the discovery leading to the inventions herein, it was not recognized that macromolecules having at least one covalently appended hydroxamate or oxime group can be used in a therapeutically effective manner to prevent or treat oxidative stress-related pathologies, including the oxidative stress-related toxicities related to an acellular hemoglobin.

### SUMMARY OF THE INVENTION

The present invention relates to methods and macromolecule compositions using hydroxamate or oxime groups covalently coupled with biological cells and macromolecules, and in particular, with albumin and acellular hemoglobin. In particular embodiments, hydroxamate or oxime groups are covalently coupled with acellular hemoglobin and compounded to provide several formulations for a red cell substitute that will possess oxidant toxin-scavenging properties. Likewise, hydroxamate or oxime groups covalently coupled with albumin or immunoglobulins endow these macromolecules with oxidant toxin detoxification properties by providing at least one hydroxamate or oxime group covalently coupled with a macromolecule possessing a variety of characterized ligand binding sites that is both stable and non-toxic in vivo, and which has a prolonged half-life in the systemic circulation. Further, hydroxamate or oxime groups covalently coupled with formed bodies, such as erythrocytes and platelets, enable oxidant toxin scavenging by providing at least one hydroxamate or oxime group covalently coupled with a cell having a lengthy half-life in the systemic circulation. Another embodiment of the invention herein involves hydroxamate or oxime groups that are covalently coupled with natural and synthetic polymers.

The invention also provides a method of preventing and treating pathologies related to the actions of oxidant toxins or oxidative stress in a mammal. The method comprises administering to a mammal a therapeutically effective amount of a macromolecule composition comprising a macromolecule coupled with at least one hydroxamate or oxime group.

### DETAILED DESCRIPTION OF THE INVENTION AND PREFERRED EMBODIMENTS THEREOF

This invention contemplates the use of stable hydroxamate and oxime groups covalently coupled with macromolecules to prevent or treat oxidative stress associated with oxidant toxin-related biological damage, including by way of example, inflammation, post-ischemic reperfusion injury, arthritis, respiratory distress syndromes, ulcerative colitis, inflammatory bowel disease, and septic shock.

Further, this invention contemplates the use of stable hydroxamate and oxime groups covalently coupled with hemoglobin to provide an oxidant detoxification function to hemoglobin-based red cell substitutes.

The term “hydroxamate group” is used herein to describe a group having the formula —C(═Q)—NHOH, where Q is O, S, or NH, said group being covalently coupled with a macromolecule. The term also encompasses embodiments in which the hydroxamate group is present as part of a thiohydroxamate, amidoxime, urea, thiourea, isourea, isothiourea, carbamate, thiocarbamate, or guanidine moiety.

The term “oxime” is used herein to describe a group having the formula —CH═NHOH, said group being covalently coupled with a macromolecule.

The term “oxidant toxin” refers to oxygen-centered free radicals such as the hydroxyl radical (HO.), a peroxyl radical (ROO.), or superoxide radical anion (O₂⁻); to ferrylhemoglobin; or to peroxynitrite (HOONO or ⁻OONO).

The term “macromolecule” is used generally herein to describe a natural or synthetic polymer. The term includes natural polymers such as proteins (e.g., hemoglobin, albumin, collagen, and insulin); polysaccharides such as dextran, cellulose, and cyclodextrin; lipopolysaccharides; liposomes; oligosaccharides, such as maltose, maltodextrin, or lactose; and oligonucleotides. Further, the term includes synthetic polymers such as nylon, poly(ethylene glycol), and povidone, together with co-polymers, branched or dendritic polymers composed at least in part of poly(ethylene glycol) [i.e., (—CH₂CH₂O—)ₙ] subunits wherein n is 3 to 100. The term excludes desferrioxamine and its metal-chelated counterparts.

The term “hemoglobin” is used generally herein to describe a hemoglobin protein that is unliganded or liganded with oxygen, carbon monoxide, or nitric oxide (that is, deoxyhemoglobin, or oxy-, carboxy-, or nitrosyl hemoglobin, respectively). The hemoglobin used with this invention may be human, recombinant, or animal in origin and is obtained and purified by known techniques. Further, the hemoglobin used with this invention may be covalently modified by intramolecular cross-linking between the globin subunits, by intermolecular cross-linking and/or polymerization, by conjugation with other macromolecules, or by a combination of these modifications. All hemoglobin formulations described herein for use with this invention are physiologically compatible and rendered suitable for infusion in the body by removal of pyrogens, endotoxin, and other contaminants.

The term “cell” is used generally herein to describe formed bodies that are present in the systemic circulation of mammals such as erythrocytes and platelets.

As used herein, the term “covalently coupled” means that the specified moieties are either directly covalently bonded to one another, or else are indirectly covalently joined to one another through an intervening moiety or moieties, such as a bridge, spacer, or linkage moiety or moieties. The term “conjugatively coupled” means that the specified moieties are covalently coupled with one another.

The term “therapeutic agent” means an agent which is therapeutically useful, e.g., an agent for the prevention, treatment, remission or attenuation of a disease state, physiological condition, symptoms, or etiological factors, or for the evaluation or diagnosis thereof. As used herein, reference to “treatment” of a patient is intended to include prophylaxis.

Other aspects, features, and modifications of the invention will be more fully apparent from the ensuing disclosure and appended claims.

In the past, low molecular weight hydroxamates such as desferrioxamine (i.e., desferoxamine mesylate) have been used therapeutically as metal chelating agents. (J. E. F. Reynolds, ed. Martindale: The Extra Pharmacopoeia. 30^{th} Ed. London: The Pharmaceutical Press, 1993, pp.676-680.) Low molecular weight hydroxamates such as bufexamac have also been used in humans as anti-inflammatory agents. (J. E. F. Reynolds, ed. Martindale: The Extra Pharmacopoeia. 30^{th} Ed. London: The Pharmaceutical Press, 1993, page 9.) In addition, low molecular weight hydroxamates have been observed to act as hypoglycemic and hypolipidemic agents. Salicohydroxamic acids and derivatives are effective antibacterial and antifungal agents. Low molecular weight hydroxamic acids are nucleophilic reactivators of sarin-inactivated chymotrypsin or acetylcholine esterase. Numerous studies show that low molecular weight hydroxamates are inhibitors of matrix metalloproteinases. (See, for example, U.S. Pat. Nos. 5,663,296; 5,665,777; 5,753,653; 5,696,147; 5,773,438; and 6,264,966.) Further, hydroxamates are inhibitors of tumor necrosis factor-alpha converting enzymes (TACE). (See, for example, U.S. Pat. Nos. 5,594,106; 5,629,285; and 6,277,885.)

Certain low molecular weight hydroxamates are also recognized as potent inhibitors of the oxidations mediated by both oxygen- and nitrogen-centered oxidant toxins that are believed to be responsible for oxidative stress-related pathologies in a mammal. For example, Davies et al. have reported that desferrioxamine inactivates superoxide [Davies M. J., Konkor R., Dunster C. A., Gee C. A., Jonas S., and Wilson R. L. Desferrioxamine (Desferal) and superoxide free radicals. Biochem. J. 1987; 246: 725-729.]. Likewise, Hartley et al. have reported that desferrioxamine is a potent lipid chain-breaking anti-oxidant in sickle cell membranes [Hartley A., Davies M. J., and Rice-Evans C. Desferrioxamine as a lipid chain-breaking antioxidant in sickle cell membranes. FEBS Lett. 1990; 264: 145-148.]. Darley-Usmar et al. have reported that desferrioxamine is a potent antioxidant and peroxyl radical scavenger [Darley-Usmar V. M., Hersey A, and Garland L. G. A method for the comparative assessment of antioxidants as peroxyl radical scavengers. Biochem. Pharmacol. 1989; 38: 1465-1469.]. Similarly, Kanner and Harel have reported that desferrioxamine inhibits membrane lipid peroxidation initiated by metmyoglobin and other peroxidizing systems [Kanner J., and Harel S. Desferrioxamine as an electron donor. Inhibition of membranal lipid peroxidation initiated by H-activated metmyoglobin and other peroxidizing systems. Free Radical Res. Commun. 1987; 3: 1-5.]. Hoe et al. have reorted that desferrioxamine inactivates the hydroxyl radical [Hoe S., Rowley D. A., and Halliwell B. Reactions of ferrioxamine and desferrioxamine with the hydroxyl radical. Chem. Biol. Interact. 1982; 41: 75-81.]. Likewise, Denicola et al. have reported that desferrioxamine (as well as other low molecular weight hydroxamates) inhibits the oxidation chemistry of peroxynitrite. [Denicola A., Souza J. M., Gatti R. M., Augusto O., and Radi R. Desferrioxamine inhibition of the hydroxyl radical-like reactivity of peroxynitrite: Role of the hydroxamic groups. Free Radical Biol. Med. 1995; 19(1): 11-19.]. Further, still other investigators have reported that the hydroxamate groups of desferrioxamine can donate hydrogen atoms or electrons to a variety of oxidizing systems including ferryl hemoglobin, ferryl myoglobin, horseradish peroxidase compound I, and activated cytochromes, with the consequent formation of the corresponding nitroxide radical. [Kanner J., and Harel S. Desferrioxamine as an electron donor. Inhibition of membranal lipid peroxidation initiated by H-activated metmyoglobin and other peroxidizing systems. Free Radical Res. Commun. 1987; 3: 1-5.] Desferrioxamine has been repeatedly used as a probe for the occurrence of the metal-catalyzed hydroxyl radical formation in biological systems.[Halliwell B. Use of desferrioxamine as a “probe” for iron-dependent formation of hydroxyl radicals. Biochem. Pharmacol. 1985; 34: 229-233.] In U.S. Pat. No. 5,506,266 Davies and Rice-Evans disclose that certain other low molecular weight hydroxamate compounds are useful for control and treatment of free radical induced damage to organs such as the heart, brain, kidney and liver.

On the basis of the literature cited above, it would be reasonable, therefore, simply to incorporate desferrioxamine (or another low molecular weight hydroxamate, urea, thiourea, isourea, isothiourea, carbamate, thiocarbamate, guanidine, or oxime) into a pharmaceutical formulation as a facile means for realizing the oxidant toxin-scavenging benefit of these potent anti-oxidants. This approach, however, fails to provide persistent therapeutic benefit for the amelioration of oxidative stress-related pathologies. Desferrioxamine, for example, is an excellent metal chelator. However, it is an active oxidant toxin-scavenger only when it as present in its unchelated form. In the plasma, desferrioxamine rapidly chelates iron and is converted to its inactive form, ferrioxamine. Further, after infusion of potentially therapeutic doses for scavenging of oxidant toxins, desferrioxamine (or another low molecular weight hydroxamate, urea, thiourea, isourea, isothiourea, carbamate, thiocarbamate, guanidine, or oxime) may be diluted by the serum to concentrations below the concentration range known to provide therapeutically effective oxidant toxin-scavenging (i.e., the therapeutic window) or may rapidly be removed from the circulation and/or excreted.

In addition, low molecular weight hydroxamates, such as desferrioxamine, may interact with cellular receptors or transfer from one body compartment to another. In view of the potent pharmacological properties of low molecular weight hydroxamates that have been described above, such interaction or transfer could initiate undesirable biological reactions and adverse physiological responses to the low molecular weight therapeutic agent.

We have now found, however, that covalently coupling hydroxamate, thiohydroxamate, amidoxime, urea, thiourea, isourea, isothiourea, carbamate, thiocarbamate, guanidine, or oxime groups with a macromolecule endows the macromolecule with the oxidant toxin-scavenging properties of a low molecular weight hydroxamate or oxime. Thus, each of the covalently coupled hydroxamate or oxime groups covalently coupled with a macromolecule in a macromolecule composition of the present invention retains the chemical reactivity to inactivate oxidant toxins that have been associated with adverse biological effects and the development of oxidative stress-related pathologies. Moreover, a macromolecule may be selected to provide a plurality of sites for covalent coupling with a hydroxamate or oxime group or both, thus endowing the macromolecule composition with the ability to inactivate oxidant toxins for greatly prolonged periods of time or at higher concentrations of these toxins. Provision must be made, however, that the sites are not grouped or co-located so as to facilitate metal chelation, since such chelation is known to inactivate the oxidant toxin-scavenging abilities of the macromolecule composition. Thus, covalently coupling a metal-chelating agent such as desferrioxamine, for example, to a macromolecule is excluded from the invention.

A macromolecule-coupled hydroxamate or oxime of the present invention offers the further advantage that it is so large that it cannot easily permeate or transfer across cell membranes. As a consequence, a macromolecule composition of the present invention that is administered by injection has a long half-life (e.g., hours or days) in the systemic circulation and demonstrates, therefore, persistent oxidant toxin-scavenging activity during its prolonged circulating half-life. For example, a particularly preferred macromolecule composition exhibiting prolonged activity, biologically safety and a desirable plasma half-life is human serum albumin (HSA) covalently coupled with hydroxamate, thiohydroxamate, amidoxime, urea, thiourea, isourea, isothiourea, carbamate, thiocarbamate, guanidine, or oxime groups. In contrast, when injected intravenously, low molecular weight hydroxamates (i.e., molecules having a molecular weight of less than about 1,000 Daltons) have very short plasma half-lives of minutes. For example, due to its molecular size and charge characteristics, desferrioxamine readily leaves the vascular space within 60 minutes after administration.

Moreover, the size and conformational features of a macromolecule composition of the present invention prevent interaction of the covalently coupled hydroxamate groups with receptors or sites on enzymes. The inability to interact with receptors or sites on enzymes largely precludes pharmacological actions other than the desirable scavenging of oxidant toxins.

Similarly, a macromolecule-coupled hydroxamate or oxime of the present invention cannot easily permeate across cell membranes into body tissues, advantageously confining its oxidant toxin-scavenging action to the biological compartment in which it is intended to provide the greatest therapeutic benefit. Thus, an orally administered, macromolecule-coupled hydroxamate or oxime of the present invention is retained in the gastrointestinal tract until excretion. The inability to cross membrane barriers such as those of the gastrointestinal tract largely precludes possibly undesirable pharmacological actions of the macromolecule composition in other body compartments.

A variety of known techniques may be used to covalently couple a hydroxamate moiety with a biomacromolecule or macromolecule. The preparative steps may require use of protecting and/or blocking groups and their subsequent removal, since these synthetic strategies may be necessary to obtain the desired product. For example, a hydroxamate may be synthesized by allowing hydroxylamine to react with an activated form of a carboxylic acid (e.g., an activated ester, an N-hydroxysuccinimidyl ester, an acid halide, an acid anhydride, and so forth). If a protected form of hydroxylamine (O-benzylhydroxylamine, for example) is used, the protective benzyl group may be removed by subsequent hydrogenation of the hydroxamate. Likewise, a hydroxamate may be prepared by allowing hydroxylamine (or a protected form of this reagent) to react with an isocyanate group, thus providing a urea embodiment of a hydroxamate, or with an isothiocyanate group, thus providing a thiourea embodiment of a hydroxamate. Similarly, a hydroxamate may be prepared by allowing an amine to react with 1-benzyloxy-3-benzyloxycarbonylthiourea in the presence of mercuric chloride and triethylamine, thus providing a guanidine embodiment of a hydroxamate. An oxime may be synthesized by allowing hydroxylamine to react with the carbonyl group of an aldehyde or ketone. The requisite functional groups (e.g., the activated carboxylic acid moiety, isocyanate group, isothiocyanate group, amino group, aldehyde or ketone moiety) may be a structural part of the macromolecule that is used as a starting material in a synthesis of a macromolecule composition of the present invention, or the preformed hydroxamate or oxime may be covalently coupled with a macromolecule to obtain a macromolecule composition of the present invention.

With respect to safety in vivo, relatively high levels of macromolecule-coupled hydroxamate groups are expected to be well tolerated, since low molecular weight hydroxamates are known to be relatively safe. For example, the oral LD₅₀ dose of the low molecular weight hydroxamate desferoxamine mesylate in the rat is 17,300 mg/kg, and the corresponding subcutaneous and intraperitoneal LD₅₀ doses of this compound in the rat are 5,500 and 330 mg/kg, respectively. Likewise, the oral LD₅₀ dose of bufexamac, another low molecular weight hydroxamate, in the rat is 3,370 mg/kg, and the corresponding subcutaneous and intraperitoneal LD₅₀ doses of this compound in the rat are >5,000 and 805 mg/kg, respectively.

Pharmaceutical compositions comprising a macromolecule-coupled hydroxamate, urea, thiourea, isourea, isothiourea, carbamate, thiocarbamate, guanidine, or oxime group as described above are also provided. Whilst it may be possible for the macromolecule compositions of the present invention to be administered as the raw chemical, it is preferable to present them as a pharmaceutical composition. According to embodiments of the present invention, a pharmaceutical composition includes one or more of the macromolecule-coupled hydroxamates, ureas, thioureas, isoureas, isothioureas, carbamates, thiocarbamates, guanidines, or oximes described above, and a pharmaceutically acceptable carrier.

The macromolecule-coupled hydroxamate, urea, thiourea, isourea, isothiourea, carbamate, thiocarbamate, guanidine, or oxime described above may be formulated for administration in a pharmaceutical carrier in accordance with known techniques. See, e.g., Remington, The Science and Practice of Pharmacy (9^{th} Ed. 1995).

In the manufacture of a pharmaceutical composition according to embodiments of the present invention, the macromolecule-coupled hydroxamate, urea, thiourea, isourea, isothiourea, carbamate, thiocarbamate, guanidine, or oxime is typically admixed with, inter alia, a pharmaceutically acceptable carrier. The carrier must, of course, be acceptable in the sense of being compatible with any other ingredients in the pharmaceutical composition and should not be deleterious to the patient. The carrier may be a solid or a liquid, or both, and is preferably formulated with the macromolecule-coupled hydroxamate, urea, thiourea, isourea, isothiourea, carbamate, thiocarbamate, guanidine, or oxime as a unit-dose formulation. The pharmaceutical compositions may be prepared by any of the well-known techniques of pharmacy, including, but not limited to, admixing the formulation components, optionally including one or more accessory ingredients.

The pharmaceutical compositions according to embodiments of the present invention include those suitable for oral, rectal, topical, inhalation (e.g., via an aerosol), buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces), intraocular, and transdermal administration. The most suitable route in any given case will depend on the nature and severity of the condition being treated and on the nature of the particular macromolecule-coupled hydroxamate, urea, thiourea, isourea, isothiourea, carbamate, thiocarbamate, guanidine, or oxime which is being used.

Pharmaceutical compositions suitable for oral administration may be presented in discrete units, such as capsules, cachets, lozenges, or tablets, each containing a predetermined amount of the macromolecule-coupled hydroxamate, urea, thiourea, isourea, isothiourea, carbamate, thiocarbamate, guanidine, or oxime; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the macromolecule-coupled hydroxamate, urea, thiourea, isourea, isothiourea, carbamate, thiocarbamate, guanidine, or oxime and a suitable carrier (which may contain one or more accessory ingredients as noted above). In general, the pharmaceutical composition according to embodiments of the present invention are prepared by uniformly and intimately admixing the macromolecule-coupled hydroxamate, urea, thiourea, isourea, isothiourea, carbamate, thiocarbamate, guanidine, or oxime with a liquid or finely divided solid carrier, or both, and then, if necessary, shaping the resulting mixture. For example, a tablet may be prepared by compressing or molding a powder or granules containing the macromolecule-coupled hydroxamate, urea, thiourea, isourea, isothiourea, carbamate, thiocarbamate, guanidine, or oxime, optionally with one or more accessory ingredients. Compressed tablets may be prepared by compressing, in a suitable machine, the macromolecule-coupled hydroxamate, urea, thiourea, isourea, isothiourea, carbamate, thiocarbamate, guanidine, or oxime in a free-flowing form, such as a powder or granules optionally mixed with a binder, lubricant, inert diluent, and/or surface active or dispersing agent(s). Molded tablets may be made by molding, in a suitable machine, the powdered compound moistened with an inert liquid binder. The tablets may optionally be coated or scored and may be formulated so as to provide slow or controlled release of the active ingredient therein.

Pharmaceutical compositions suitable for buccal (sub-lingual) administration include lozenges comprising the macromolecule-coupled hydroxamate, urea, thiourea, isourea, isothiourea, carbamate, thiocarbamate, guanidine, or oxime in a flavored base, usually sucrose and acacia or tragacanth; and pastilles comprising the macromolecule-coupled hydroxamate, urea, thiourea, isourea, isothiourea, carbamate, thiocarbamate, guanidine, or oxime in an inert base such as gelatin and glycerin or sucrose and acacia.

Pharmaceutical composition according to embodiments of the present invention suitable for parenteral administration comprise sterile, aqueous and non-aqueous injection solutions of the macromolecule-coupled hydroxamate, urea, thiourea, isourea, isothiourea, carbamate, thiocarbamate, guanidine, or oxime, which preparations are preferably isotonic with the blood of the intended recipient. These preparations may contain anti-oxidants, buffers, baceriostats, and solutes which render the composition isotonic with the blood of the intended recipient. Aqueous and non-aqueous sterile suspensions may include suspending agents and thickening agents. The compositions may be presented in unit-dose or multi-dose containers, for example, sealed ampoules and vials, and may be stored in a freeze-dried (lyophilized) condition requiring only the addition of the sterile liquid carrier, for example, saline or water for injection, immediately prior to use. Extemporaneous injection solutions and suspensions may be prepared from sterile powders, granules, and tablets of the kind previously described. For example, an injectable, stable, sterile composition comprising a macromolecule-coupled hydroxamate, urea, thiourea, isourea, isothiourea, carbamate, thiocarbamate, guanidine, or oxime in a unit dosage form in a sealed container may be provided.

Pharmaceutical compositions suitable for rectal administration are preferably presented as unit-dose suppositories. These may be prepared by admixing the macromolecule-coupled hydroxamate, urea, thiourea, isourea, isothiourea, carbamate, thiocarbamate, guanidine, or oxime with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Pharmaceutical compositions suitable for transdermal administration may be presented as discrete patches adapted to remain in intimate contact with the epidermis of the recipient for a prolonged period of time. Compositions suitable for transdermal administration may also be delivered by iontophoresis (see, for example, Pharmaceutical Research 3(6): 318 (1986)) and typically take the form of an optionally buffered aqueous solution of the macromolecule-coupled hydroxamate, urea, thiourea, isourea, isothiourea, carbamate, thiocarbamate, guanidine, or oxime. Suitable formulations comprise citrate or bis-tris buffer (pH 6) or ethanol/water and contain from 0.1 to 0.2 M active ingredient.

It should be understood that in addition to the ingredients particularly mentioned above, the formulations of this invention may include other agents conventional in the art having regard to the type of formulation in question, for example those suitable for oral administration may include flavoring agents.

Preferred unit dosage formulations are those containing an effective dose, as hereinbelow recited, or an appropriate fraction thereof, of the active ingredient.

According to other embodiments of the present invention, methods of treating a patient in need of such treatment include administering to the patient an effective amount of a macromolecule composition comprising a macromolecule covalently conjugated with one or more hydroxamate, urea, thiourea, isourea, isothiourea, carbamate, thiocarbamate, guanidine, or oxime groups as described above. The therapeutically effective amount of any macromolecule-coupled hydroxamate, urea, thiourea, isourea, isothiourea, carbamate, thiocarbamate, guanidine, or oxime, the use of which is in the scope of the present invention, will vary somewhat from one macromolecule composition to another, and from patient to patient, and may depend on factors such as the age and condition of the patient and the route of delivery. Such dosages can be determined in accordance with routine pharmacological procedures known to those skilled in the art. As a general proposition, a therapeutically effective dose of macromolecule-coupled hydroxamate, urea, thiourea, isourea, isothiourea, carbamate, thiocarbamate, guanidine, or oxime will be the weight of active pharmaceutical ingredient per kg of the patient's body weight (i.e., mg/kg) that is useful for the prevention, prophylaxis, treatment, remission or attenuation of a disease state, physiological condition, symptoms, or etiological factors, or for the evaluation or diagnosis thereof. The duration of treatment depends on the type of condition being treated and may be for as long as the life of the patient.

The present invention will now be described with reference to the following examples. It should be appreciated that these examples are for the purposes of illustrating aspects of the present invention, and do not limit the scope of the invention as defined by the claims.

### EXAMPLES

### Example 1

Hydroxamate-PEG, a preferred embodiment. Mercuric chloride (0.9 g, 3.2 mmol) is added to a well stirred solution of 1-benzyloxy-3-benzyloxycarbonylthiourea (1.0 g, 3.2 mmol), α,ω-bis(amino)poly(ethylene glycol) (molecular weight about 3500; 4.9 g, 1.4 mmol), and triethylamine (4.2 g, 42 mmol) in 4 mL of dimethylformamide. The reaction mixture is stirred for 8 h, diluted with 20 mL of ethyl acetate, and filtered. The filtrate is washed twice with 20 mL of water and 20 mL of brine. The organic phase is dried over anhydrous sodium sulfate. Filtration and concentration provides a dark oil which is purified on a silica gel column (eluted with ethyl acetate). About 34% of the desired product, α,ω-bis(N-hydroxyguanyl)poly(ethylene glycol) is thus obtained.

### Example 2

Hydroxamate-PEG, a preferred embodiment. A solution of the bis(N-hydroxysuccinimide) activated ester of poly(ethylene glycol) bis(carboxymethyl) ether (molecular weight about 750; 75 mg, 0.1 mmol) in tetrahydrofuran is treated with hydroxylamine hydrochloride (15 mg, 0.24 mmol) and triethylamine (4.2 g, 42 mmol). The reaction mixture is stirred for 8 h, diluted with 20 mL of ethyl acetate, and filtered. The filtrate is washed successively with 20 mL of 0.1 N hydrochloric acid, 20 mL of water, 20 mL of 0.1 N sodium bicarbonate, and 20 mL of brine. The organic phase is dried over anhydrous sodium sulfate. Filtration and concentration provides a pale yellow oil which is purified on a silica gel column (eluted with ethyl acetate). About 45% of the desired product, alpha,omega-bis-PEG-amidoxime, is thus obtained.

### Example 3

Branched PEG having covalently coupled hydroxamate groups, a preferred embodiment. Multi-armed PEG polymers are disclosed in U.S. Pat. Nos. 6,046,305, 5,932,462 and 5,643,575. A 3-armed PEG in which each arm terminates in a carboxyl group is prepared by allowing 0.3 moles of α-amino-ω-(methoxycarbonylethyl)-PEG to react with 0.1 mole trimesyl chloride in acetonitrile solution containing a 5-molar excess of triethylamine. The reaction mixture is stirred for 8 h, diluted with ethyl acetate, and filtered. The filtrate is washed successively with 0.1 N hydrochloric acid, water, sodium bicarbonate, and brine. The organic phase is dried over anhydrous sodium sulfate. Filtration and concentration provides an oil which is purified on a silica gel column. The desired product, a 3-armed PEG in which each arm terminates in a methoxycarbonyl group, is thus obtained.

The methyl esters at the end of each PEG segment are hydrolyzed by exposure to 0.1 N sodium hydroxide. The solution is acidified with 0.1 N HCl, and extracted several times with ethyl acetate. The combined organic extracts are dried with anhydrous magnesium sulfate, filtered, and concentrated under vacuum. The residual oil is dissolved in acetonitrile and treated with 3.3 equivalents of disuccinimidyl carbonate in the presence of triethylamine, thus providing an N-hydroxysuccinimidyl activated ester at the terminus of each arm of the 3-armed PEG.

### Example 4

Salicohydroxamate-PEG, a particularly preferred embodiment. 5-Aminosalicylic acid is converted to the corresponding 5-aminosalicohydroxamate by conversion to an activated ester and reaction with hydroxylamine. Three equivalents of the resulting material are allowed to react with 1 equivalent of the N-hydroxysuccinimidyl-activated 3-armed PEG reagent prepared in Example 3 in acetonitrile solution containing triethylamine. The reaction is stirred at room temperature for 24 h and filtered. Solvent is removed under reduced pressure, and the residual solid is purified by chromatography on silica gel. Salicohydroxamate-PEG, a 3-armed PEG hydroxamate, is thus obtained.

### Example 5

N-[ω-(N-hydroxyacetamido)-PEG4]proline N-hydroxysuccinimidyl ester. ω-Bromo-PEG4-carboxylic acid is converted to an activated ester by reaction with disuccinimidyl carbonate in dimethylformamide solution containing triethylamine. The activated ester is allowed to react with 1 equivalent of hydroxylamine in dimethylformamide solution containing triethylamine. The product, 1-bromo-ω-(N-hydroxyacetamido)-PEG4 is allowed to react with L-proline ethyl ester in acetonitrile solution. The N-[ω-(N-hydroxyacetamido)-PEG4]proline ethyl ester thus obtained is hydrolyzed to the corresponding acid by treatment with 0.1 N NaOH. The reaction mixture is acidified with 0.1 N HCl and extracted with ethyl acetate. The combined organic extracts are dried with anhydrous magnesium sulfate, filtered, and concentrated under vacuum. The desired product, N-[ω-(N-hydroxyacetamido)-PEG4]proline, is purified by chromatography on silica gel. A dimethylformamide solution of this material is treated with disuccinimidyl carbonate in the presence of triethylamine. After workup, the desired product N-[ω-(N-hydroxyacetamido)-PEG4]proline N-hydroxysuccinimidyl ester, is obtained.

### Example 6

Hydroxamate-substituted human serum albumin, a particularly preferred embodiment. An aqueous solution of human serum albumin (HSA) (665 mg, 10 μmol) in borate buffer, pH 9, is treated with an acetonitrile solution of N-[ω-(N-hydroxyacetamido)-PEG4]proline N-hydroxysuccinimidyl ester (100 μmol). The reaction mixture is stirred at room temperature for 24 h. After this time, the mixture is subjected to dialysis using a membrane with a 30,000 Dalton molecular weight cutoff. The degree of amino group substitution is determined by trinitrobenzenesulfonic acid (TNBS) assay and mass spectrometry, and the protein concentration by Biuret method. A molar ratio of hydroxamate-PEG conjugate to albumin of about 5 is found. The desired product is also obtained by using an organic solvent such as DMSO or DMF.

### Example 7

Hydroxamate-PEG-Hemoglobin, a particularly preferred embodiment. An aqueous solution of purified human hemoglobin (650 mg, 10 μmol) in phosphate buffered saline, pH 7.4, is treated with a solution of N-[ω-(N-hydroxyacetamido)-PEG4]proline N-hydroxysuccinimidyl ester (100 μmol) in a minimum volume of 1 M HEPES. The reaction mixture is stirred at room temperature for 24 h. After this time, the mixture is subjected to dialysis using a membrane with a 30,000 Dalton molecular weight cutoff. The degree of amino group substitution is determined by mass spectrometry, and the protein concentration by UV-visible spectrophotometry. A molar ratio of hydroxamate-PEG conjugate to hemoglobin of about 5 is found.

The present invention has been described herein with reference to its preferred embodiments. These embodiments do not serve to limit the invention, but are set forth for illustrative purposes. The scope of the invention is defined by the claims that follow.

I claim:

1. A macromolecule composition comprising a macromolecule covalently coupled with at least one group having the formula
—C(═Q)—NH—OH,
where Q is O, NH, or S, a thiohydroxamate, amidoxime, urea, thiourea, isourea, isothiourea, carbamate, thiocarbamate, or guanidine moiety, thereby imparting oxidant-toxin scavenging capabilities to the composition.

2. The macromolecule composition according to claim 1, wherein the macromolecule is a protein.

3. The macromolecule composition according to claim 1, wherein the macromolecule is albumin or hemoglobin.

4. The macromolecule composition according to claim 1, wherein the macromolecule is a polysaccharide.

5. The macromolecule composition according to claim 1, wherein the macromolecule is nylon, poly(ethylene glycol), povidone, or a co-polymer composed at least in part of (—CH₂CH₂O—)ₙ where n is 3 to 100.

6. The macromolecule composition according to claim 1, wherein the macromolecule is a cell.

7. The macromolecule composition according to claim 6, wherein the cell is selected from the group consisting of erythrocytes, platelets, erythrocyte ghosts, and endothelial cells.

8. The macromolecule composition according to claim 1, wherein Q is O.

9. The macromolecule composition according to claim 1, wherein Q is S.

10. The macromolecule composition according to claim 1, wherein Q is NH.

11. A macromolecule composition comprising a macromolecule covalently coupled with at least one group having the formula
—C(═Q)—NH—OH,
where Q is O, NH, or S, a thiohydroxamate, amidoxime, urea, thiourea, isourea, isothiourea, carbamate, thiocarbamate, or guanidine moiety, thereby imparting oxidant-toxin scavenging capabilities to the composition, wherein the oxidant toxin is a hydroxyl radical (HO.).

12. A macromolecule composition comprising a macromolecule covalently coupled with at least one group having the formula
—C(═Q)—NH—OH,
where Q is O, NH, or S, a thiohydroxamate, amidoxime, urea, thiourea, isourea, isothiourea, carbamate, thiocarbamate, or guanidine moiety, thereby imparting oxidant-toxin scavenging capabilities to the composition, wherein the oxidant toxin is a peroxyl radical.

13. A macromolecule composition comprising a macromolecule covalently coupled with at least one group having the formula
—C(═Q)—NH—OH,
where Q is O, NH, or S, a thiohydroxamate, amidoxime, urea, thiourea, isourea, isothiourea, carbamate, thiocarbamate, or guanidine moiety, thereby imparting oxidant-toxin scavenging capabilities to the composition, wherein the oxidant toxin is peroxynitrite.

14. A macromolecule composition comprising a macromolecule covalently coupled with at least one group having the formula
—C(═Q)—NH—OH,
where Q is O, NH, or S, a thiohydroxamate, amidoxime, urea, thiourea, isourea, isothiourea, carbamate, thiocarbamate, or guanidine moiety, thereby imparting oxidant-toxin scavenging capabilities to the composition, wherein the oxidant toxin is ferryl hemoglobin.

15. A macromolecule composition comprising a macromolecule covalently coupled with at least one group having the formula
—C(═Q)—NH—OH,
where Q is O, NH, or S, a thiohydroxamate, amidoxime, urea, thiourea, isourea, isothiourea, carbamate, thiocarbamate, or guanidine moiety, thereby imparting oxidant-toxin scavenging capabilities to the composition, wherein the oxidant toxin is the superoxide radical-anion.

16. A macromolecule composition comprising a macromolecule covalently coupled with at least one having the formula
—CH═NH—OH.

17. The macromolecule composition according to claim 16, wherein the macromolecule is a protein.

18. The macromolecule composition according to claim 16, wherein the macromolecule is albumin or hemoglobin.

19. The macromolecule composition according to claim 16, wherein the macromolecule is a polysaccharide.

20. The macromolecule composition according to claim 16, wherein the macromolecule is nylon, poly(ethylene glycol), povidone, or a co-polymer composed at least in part of (—CH₂CH₂O—)ₙ where n is 3 to 100.

21. A red cell substitute comprising an acellular hemoglobin composition in a physiologically compatible solution, wherein said hemoglobin composition comprises an acellular hemoglobin stabilizingly and covalently coupled with at least one group having the formula
—C(═Q)—NH—OH,
wherein Q is O, NH, or S, a thiohydroxamate, amidoxime, urea, thiourea, isourea, isothiourea, carbamate, thiocarbamate, or guanidine moiety.

22. The red cell substitute according to claim 20, wherein Q is O.

23. The red cell substitute according to claim 20, wherein Q is S.

24. The red cell substitute according to claim 20, wherein Q is NH.

25. A red cell substitute comprising an acellular hemoglobin composition in a physiologically compatible solution, wherein said hemoglobin composition comprises an acellular hemoglobin stabilizingly and covalently coupled with at least one group having the formula
—CH═NH—OH.

26. A method for the prevention or treatment of an oxidative stress-related pathology in a patient, comprising the step of administering to said patient a therapeutically effective amount of a macromolecule composition comprising a macromolecule covalently coupled with at least one group having the formula:
—C(═Q)—NH—OH,
where Q is O, NH, or S, a thiohydroxamate, amidoxime, urea, thiourea, isourea, isothiourea, carbamate, thiocarbamate, or guanidine moiety.

27. The method according to claim 26, wherein the macromolecule is albumin or hemoglobin.

28. The method according to claim 26, wherein the macromolecule is a polysaccharide.

29. The method according to claim 26, wherein the macromolecule is nylon, poly(ethylene glycol), povidone, or a co-polymer composed at least in part of (—CH₂CH₂O—)ₙ where n is 3 to 100.

30. The method according to claim 26, wherein the oxidative stress-related pathology is inflammation, post-ischemic reperfusion injury, arthritis, respiratory distress syndrome, ulcerative colitis, inflammatory bowel disease, or septic shock.

31. A method for the prevention or treatment of an oxidative stress-related pathology in a patient, comprising the step of administering to said patient a therapeutically effective amount of a macromolecule composition, wherein said macromolecule composition comprises a macromolecule covalently coupled with at least one oxime group.
